(19) United States
(12) Patent Application Publication
(10) Pub. No.: US 2005/0159795 A1
(43) Pub. Date: Jul. 21, 2005

(54) Light and ion therapy apparatus and method

(76) Inventors: Kent W. Savage, American Fork, UT (US); Steven D. Powell, Orem, UT (US)

Correspondence Address: TRASK BRITT, P.O. BOX 2550, SALT LAKE CITY, UT, 84110, US

(21) Appl. No.: 10/925,231

(22) Filed: Aug. 23, 2004

Related U.S. Application Data
(60) Provisional application No. 60/496,855, filed on Aug. 21, 2003.

(51) Int. Cl. A61N 1/00

(52) U.S. Cl. 607/88

(57) ABSTRACT

A light and ion therapy apparatus for delivering ocular light to a subject to treat disorders that are responsive to ocular light therapy, includes a light therapy unit for delivering to an eye of a subject light having an intensity of at least about 10,000 lux at a distance of about six inches or greater, and an ion therapy unit integral with the light therapy unit and independently operated therefrom for delivering to the subject a therapeutic dosage of high-density negative ions. A light and ion therapy method for delivering light to an eye of a subject to treat disorders that are responsive to ocular light therapy, includes delivering to an eye of a subject light having an intensity of at least about 10,000 lux at a distance of about six inches or greater, and independently delivering to the subject a therapeutic dosage of high-density negative ions.

### CROSS-REFERENCE TO RELATED APPLICATION

Under the provisions of 35 U.S.C. § 119(e), priority is claimed from U.S. Provisional Application Ser. No. 60/496,855, filed on Aug. 21, 2003.

### BACKGROUND OF THE INVENTION

### Field of the Invention

This application relates to light and ion therapy apparatus and methods. More particularly, this application concerns apparatus and methods for providing light and ion therapy to a subject to achieve circadian (biological time clock) rhythm adjustments, and treat seasonal affective disorder, depression, chronobiological disorders and other related disorders or problems.

High-intensity (greater than 2,500 lux) light therapy systems have been widely used to treat circadian rhythm disorders, seasonal affective disorder and other such problems by delivering light through the eyes of a subject. Light therapy is usually received through photoreceptors in the eyes, travels through certain neurological pathways to the suprachaismatic nuclei (SCN) to eventually interact with proteins in the blood stream. This type of therapy stimulates and/or regulates the production of certain hormones and other substances. For example, it may aid in the production of seritonin and norepinephrine. It also regulates and suppresses the production of melatonin.

It has been found that a variety of physical disorders may be linked to biological time clock problems. For example, studies in chronobiology indicate that problems dealing with hormonal imbalances, heart disease, and chronic fatigue may be connected to a subject's personal circadian rhythm. Adjustments in the amount and timing of exposure to therapeutic light, coupled with time adjustments in sleeping, eating, exercise and medication, may substantially improve a subject's resistance to and ability to recover from many physical maladies.

Various devices have been used to attempt to provide the necessary light intensity and color spectrum, similar to daylight. In some cases fluorescent lights are used because they can provide an effective spectrum of light, a broad and comfortable field of light, and are longer lasting than incandescent lamps. However, the high intensities of light needed for such treatments require relatively large-sized lamps and other components. Thus, many commercial light therapy units have been large, bulky and cumbersome.

More recently, smaller, lighter-weight light therapy units have been produced. An example is shown in U.S. Pat. No. 6,488,698 (Hyman). However, such devices have not been able to provide an effective dosage of light, except at very close range. This prevents the subject from being able to do other tasks while receiving light therapy.

Another type of therapy, called high-density negative ion therapy, (referred to herein as “ion therapy”) has been developed for the purpose of treating atypical depressive disorders and seasonal affective disorders by administering high-density negative ions produced by an ion generator. This type of ion generator is not used for cleaning the air but, rather, is applied for the purpose of treating a subject with beneficial negatively charged ions. An example is shown in U.S. Pat. No. 5,533,527 (Terman), in which the subject is treated with high-density negative ions for about 30 minutes per day for successive days.

The high-density negative ions produced by ion therapy tend to be absorbed into the body through the skin and/or lungs to eventually reach the blood stream. It is believed that negative ions tend to cause the SCN to shift to an active phase, further suppressing withdrawal and depressive hormones. Ion therapy may also affect the prefrontal cortex in the brain, associated with mood and behavior. Some theorists believe that positive ions cause a synaptic breakdown in the brain. Administering a high dosage of negative ions is believed to tend to break down the synaptic roadblocks and allow these pathways in the brain to function properly.

Recognizing the beneficial value of both light therapy and negative ion therapy, some units have been developed that provide doses of both light and negative ions at the same time, in order to treat the disorders mentioned above. However, there have been problems with such devices providing sufficient intensity of light and densities of negative ions in a combination that would be beneficial for treatment.

### SUMMARY OF THE INVENTION

In one implementation, a light and ion therapy apparatus for delivering light to an eye of a subject to treat disorders that are responsive to ocular light therapy comprises a light therapy unit for delivering to an eye of the subject light having an intensity of at least about 10,000 lux at a distance of about six inches or more, and a ion therapy unit integral with the light therapy unit and operated independently therefrom, for delivering to the subject a therapeutic dosage of high-density negative ions.

In another implementation, a method of light and ion therapy for delivering light to an eye of a subject to treat disorders that are responsive to ocular light therapy comprises delivering to an eye of the subject light having an intensity of at least about 10,000 lux at a distance of about six inches or more, and independently delivering to the subject a therapeutic dosage of high-density negative ions.

Other features and advantages of the present invention will become apparent to those of ordinary skill in the art through consideration of the ensuing description, the accompanying drawings, and the appended claims.

### BRIEF DESCRIPTION OF THE SEVERAL VIEWS OF THE DRAWINGS

In the drawings, which depict various features of exemplary embodiments of the present invention:

FIG. 1 is a pictorial depiction of one implementation of the light and ion therapy device providing treatment to a subject;

FIG. 2 is a perspective view of the light and ion therapy device of FIG. 1;

FIG. 3 is a front plan view of the cover for the device shown in FIG. 2;

FIG. 4 is a side plan view of the device shown in FIG. 2;

FIG. 5 is a perspective view of the device of FIG. 2 with the front cover removed;

FIG. 6 is a partial cross-sectional view of the device shown in FIG. 5;

FIG. 7 is an illustration of a light tube and reflector according to the device shown in FIG. 5;

FIG. 8 is a perspective view of another light therapy and ion therapy embodiment of the present invention;

FIG. 9 is a partial cross-sectional view of the embodiment shown in FIG. 8; and

FIG. 10 is a perspective view of another light therapy and ion therapy embodiment of the present invention.

### DETAILED DESCRIPTION OF THE INVENTION

In one embodiment, the light and ion therapy device of the present disclosure delivers a full spectrum of light to the subject at the level of about 10,000 lux or more. Alternately, therapeutic light having wavelengths in the blue part of the spectrum (about 430-490 nanometers), might be delivered at a lower power level, such as 2.2×10⁻⁴ mw/cm² or more. At these intensity levels, a therapeutic dosage of light is delivered during a period of about 15 to 25 minutes. The desired intensity of light may be delivered to the subject at a distance of about six inches or more. In the present embodiment, one preferred range of distance was about six to 15 inches. In another embodiment, another preferred range of distance was about six to 24 inches. The light and ion therapy device of the present disclosure also delivers high-density negative ions in a concentration sufficient to provide a therapeutic dosage of negative ion therapy to the subject.

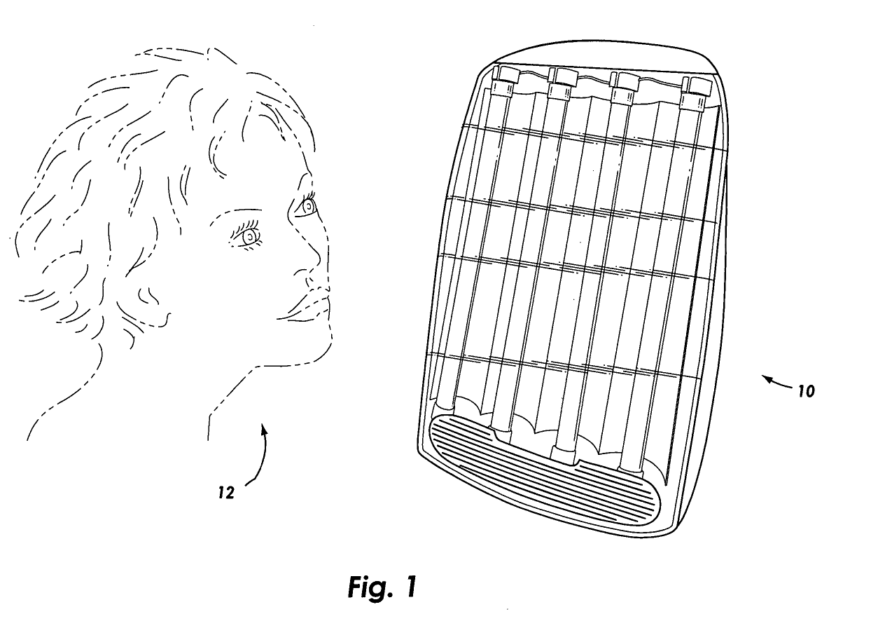

Referring to FIG. 1, a light and ion therapy device 10 shown that is portable, at about six pounds, to be placed in any desired location so as to provide a therapy session quickly and conveniently. The subject 12 is able to receive a desirable amount of about 10,000 lux of light therapy at a comfortable distance of about six inches or more to accentuate comfort and to enable other tasks to be carried out by the subject during therapy. The light intensity provided by the embodiments of the current invention enables administering a therapeutic dose of light far beyond 6 inches. The subject may comfortably receive light therapy at about 15 inches, and therapy may be applied at greater distances, such as 36 inches or more. It is understood that therapeutic light may be delivered to a subject directly into the eyes and, to a lesser degree, through the subject's eyelids to the eyes.

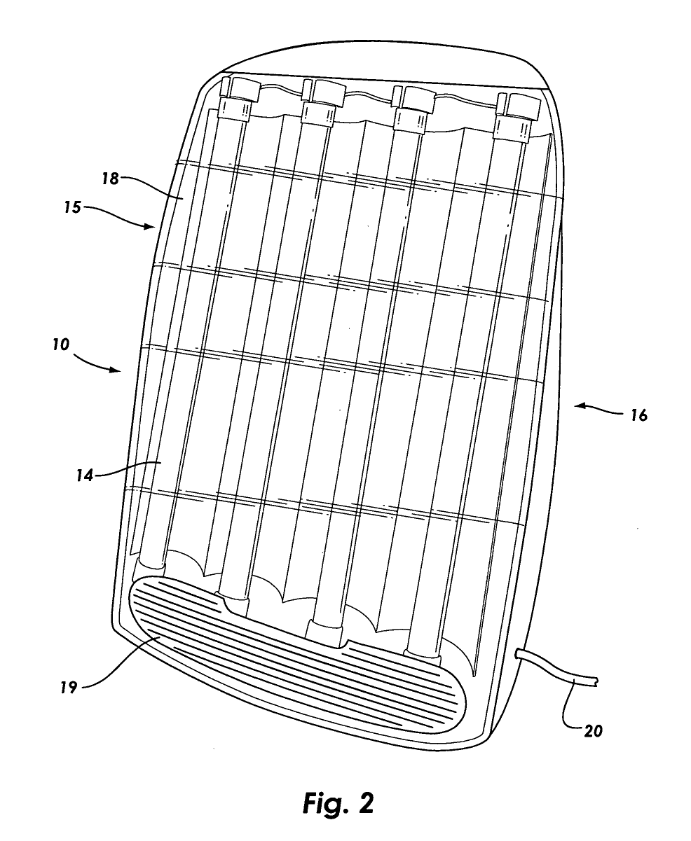

FIG. 2 shows light and ion therapy device 10 in perspective view. Four fluorescent lamps 14 are disposed within a case or housing 16 that is structured to stand substantially upright, tilted slightly from horizontal. A front cover 15 of case 16 has a transparent diffuser lens plate 18 through which lamps 14 transmit diffused light. A grill 19 is provided at the bottom of cover 16, through which ions are administered to the subject. An electric power cord 20 provides standard electrical current to device 10. Housing 16 may be made of a molded polymer and be about 13.5 inches high, about 9 inches wide and about 5 inches deep.

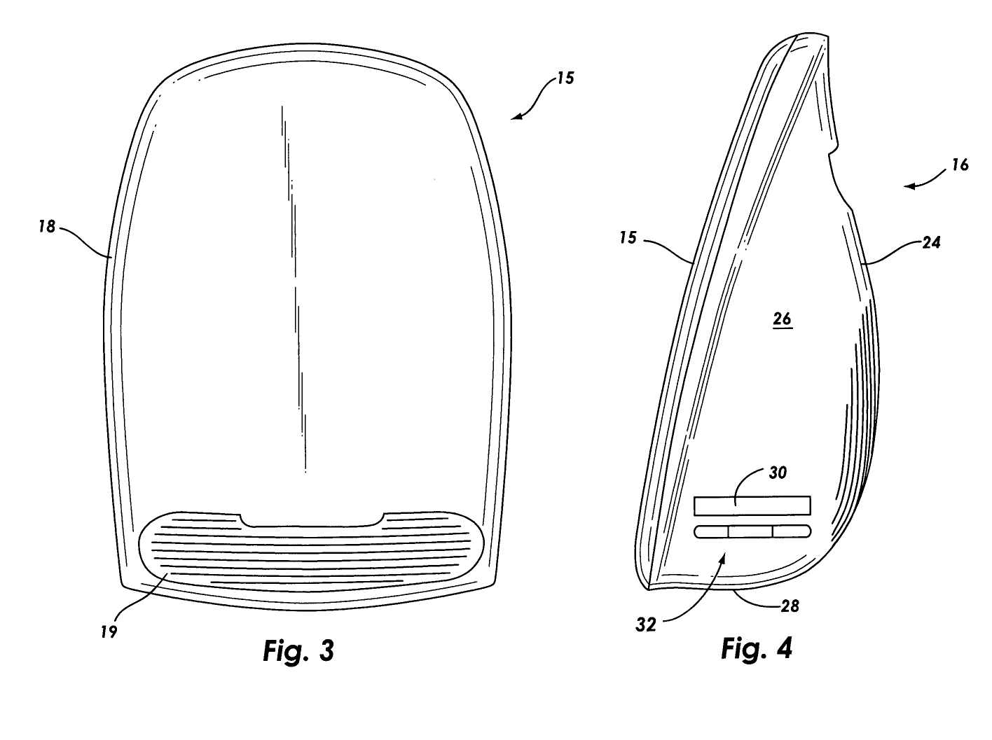

FIG. 3 shows a frontal view of the cover 15 of the light therapy unit 10, comprising the lens 18 and grill 24, through which ions are administered to a subject. FIG. 4 is a side view of housing 16, showing cover 15, a curved back 24 and a side 26. A base 28 supports housing 16 in an upright position for administering light therapy to a subject. Lens 18 may be a very thin (1/16 inch) unit that may be textured and made of clear acrylic. The diffraction of the light passing through the texture of the lens softens the high-intensity light and allows a more uniform treatment field.

A display unit 30 is provided on the side for showing the time and other parameters. Three input buttons 32 below display unit 30 are used to input data for the control of timers for the light output and ion output devices.

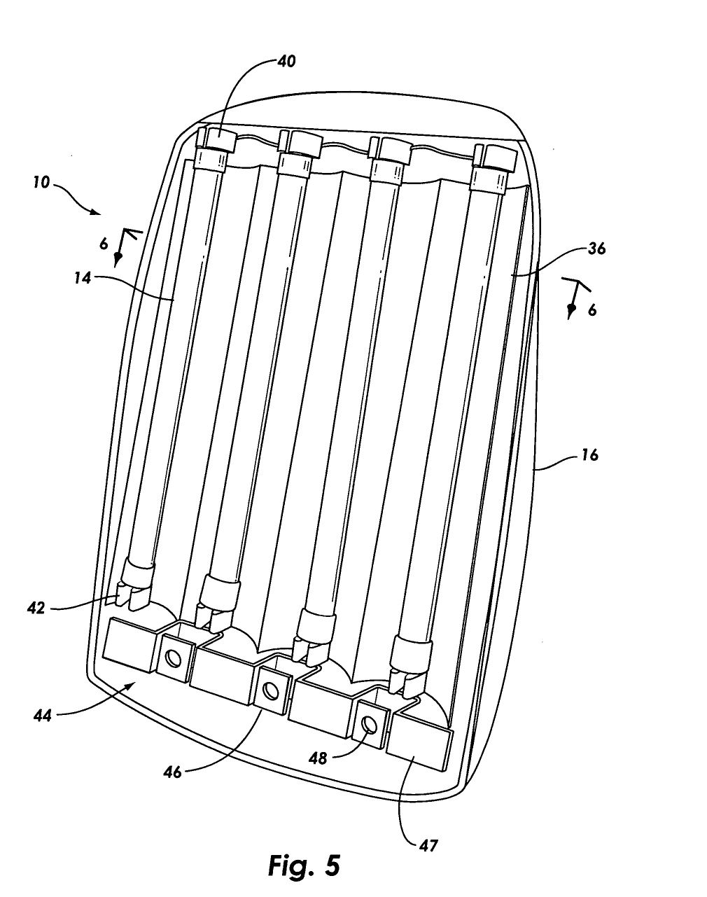

FIG. 5 shows the unit 10 with the front cover 15 removed, to expose lamps 14. Each lamp 14 is a fluorescent tube, emitting about 10,000 lux or more, full spectrum, at a color rating of about 5,500 Kelvin. Lamps 14 are disposed in conventional socket connectors 40 and 42. Electrical wiring behind the lamps (not shown) connects each lamp 14 to a ballast (not shown) which is, in turn, connected to the power cord 20. The ballast may be a conventional electronic or magnetic ballast, known in the art. A curved plate 36 is disposed behind the lamps to provide substantial additional light by reflection.

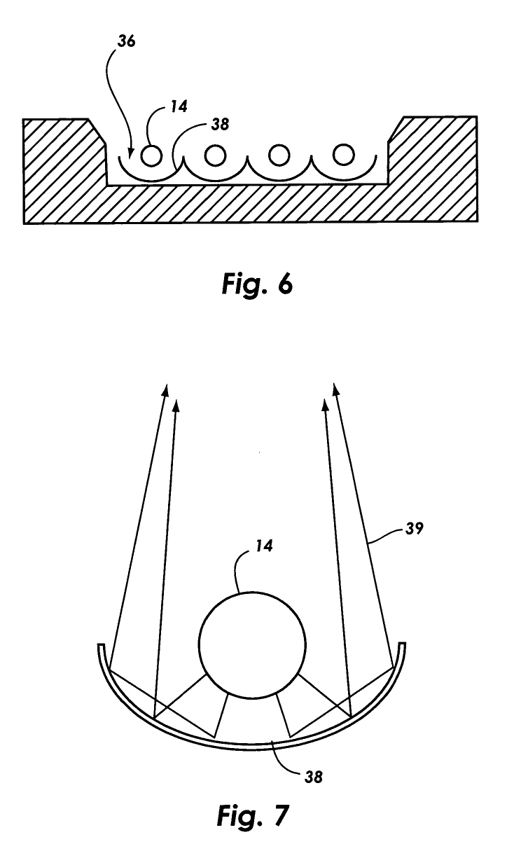

FIG. 6 is a cross-section of FIG. 5, in which curved reflector member 36 is shown in greater detail. Reflectors 36 is formed to enhance the light output by focusing the light emitted by lamp tubes 14. Reflector 36 comprises four curved reflector portions 38 each having a generally curved shape. As used herein, the term “curved” includes, but is not limited to, a substantially parabolic curve and a series of bends that roughly follow a curved line. Reflector 36 may be comprised of thin highly-reflective metal, such as polished or coated aluminum to achieve reflectivity of 90% or more. The curved portions 38 are curved sufficiently to enhance the amount of light directed to the subject 12 (FIG. 1) from the tubes 14. In the present embodiment, the amount of curvature in the curved portions 38 corresponds to directing at least about 10,000 lux of light to the subject 12 at a distance of about six to nine inches from the device 10.

FIG. 7 shows the reflective angles of light reflected light according to the embodiment shown in FIG. 6. Each lamp or tube 14 may be positioned approximately at the focal point of one of the curved portions 38 in the curved reflector 36. Thus, with the lamp 14 at the focal point of the curved portion 38, the rays of light 39 reflect from the surface of the curved portion 38 and are directed through the lens 18, shown in FIG. 2, towards the subject 12 (FIG. 1) to provide enhanced reflectivity of the generated light.

Referring again to FIG. 5, an ion emitter 44 is disposed at the base of the housing 16 to project ions through the grill 19 shown in FIGS. 1-3. Ion emitter 44 is a conventional ion emitter having three separate emitter plates 46 disposed on a common mount 47. Each emitter plate 46 has a circular aperture 48 therein. A needle point (not shown) is disposed in or behind each aperture 48 for generating the ions. Ion emitter 44 projects negative ions through the grill 19 to the subject being treated. By way of example, emitter 44 may produce about 120 trillion negative ions per second to provide a high-density of negative ions (about 2×10⁴ ions/cm³ or more) in the vicinity of the therapy subject 12 (FIG. 1). In said example, a therapeutic dosage of negative ions was delivered in a period of 20 minutes or less. A preferred ion generation unit is provided in the Sun Touch Plus model marketed by Apollo Light Systems of Orem, Utah.

Another embodiment of the present application with the same apparatus as in FIGS. 1-5 may use a lamp that either provides exclusively blue light or emits a broader spectrum than just blue light but has an emphasized intensity for the blue light range. As used herein, the terms “blue light” and “blue light range” mean light having a wavelength in the range of about 430 to 490 nanometers. Light in the blue light range has been found to be particularly beneficial in light therapy applications.

Another embodiment comprises the use of lamps that provide exclusively green light or emits a broader spectrum than just green light but has an emphasized intensity for the green light range alone or for a combination of the green and blue light ranges. As used herein, the terms “green light” and “green light range” mean light having a wavelength in the range of about 490 to 520 nanometers. Light in the green light range has been found to be particularly beneficial in light therapy applications.

Yet another embodiment may use a broad spectrum lamp combined with a filtering lens that together provide light to a subject that is either exclusively blue light or has a higher intensity of blue light than other light within the spectrum.

In still another embodiment with the same apparatus shown in FIGS. 1-5, lamps or tubes may be selected that emit a full spectrum of visible light but does not emit any significant amount of ultra-violet light. Optionally, a filter may be included to filter out undesired wavelengths of electromagnetic radiation or to facilitate selection of the wavelength or wavelengths that pass through the lens. In this regard, the lamps or tubes or the lens may be made of a material that would filter out ultra-violet light or other undesired wavelengths of electromagnetic radiation. Alternatively, a filter may be disposed between the lamps or tubes and the lens or external to the lens. Such filters may be replaceable, permitting the subject or another individual to select the wavelength or wavelengths of electromagnetic radiation that are to be filtered before reaching the subject.

The combination of providing light therapy and ion therapy together, as shown above, has synergistic results for the subject, in several respects. First, by applying the two therapies together, the time for therapy treatment is substantially shortened. For example, it is known that a therapeutic dosage of high-density negative ions must be applied over a time period of 30 minutes or more to achieve the desired effects. Likewise, a therapeutic dose of light therapy might require exposure to light for a period of 30 minutes or more. Applying both light therapy and ion therapy together enables a subject to reduce the period of therapy treatment to about 15-20 minutes. Since many people have extremely busy schedules, this shortening of therapy time is a substantial advantage.

In addition, light therapy and ion therapy each provide different therapeutic effects that tend to supplement and/or reinforce each other. As mentioned above, light therapy is received through the eyes and interacts with the suprachaismatic nuclei (SCN) to stimulate and/or regulate the production of certain hormones and other substances. Ion therapy is absorbed through the skin and/or the lungs to cause the SCN to shift into an active phase. Ion therapy may also affect the prefrontal cortex in the brain, associated with mood and behavior. Thus, the two types of therapy are different and tend to work together and provide synergistic results.

Furthermore, the physiological differences in various subjects tend to provide different reactions to light therapy and ion therapy. Thus, one subject may be more receptive to light therapy treatment and less susceptible to ion therapy than the norm. Others may find that ion therapy treatment tends to be more beneficial than light therapy. By providing both therapies in the same unit, each subject can find a balance of light and ion therapies that will provide the best results for him/her. Thus, using the unit of the present invention, each user can develop a personalized light and ion therapy regimen.

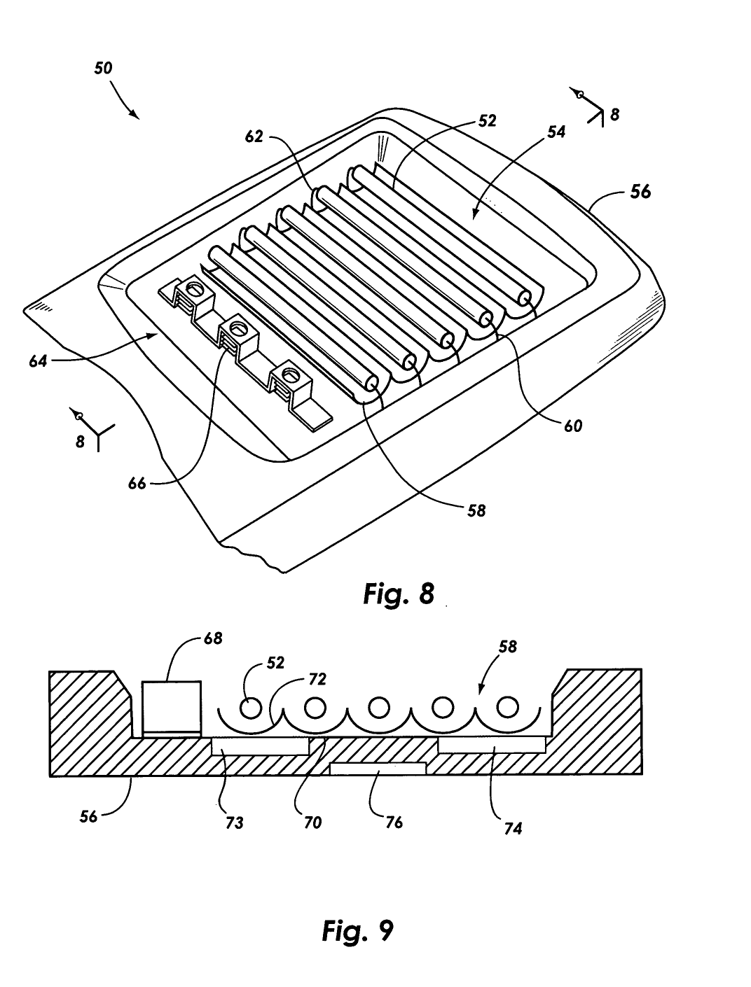

FIGS. 8 and 9 show another embodiment comprising a light therapy unit 50, a hand-held, battery powered, ocular therapy device that incorporates a unique type of fluorescent light source called cold cathode fluorescent lamps, also referred to as CCFL. CCFL tubes are usually low-pressure lamps, for example, using mercury vapor, and having a very small diameter (for example, 2 to 3 mm) and short length (for example, 50 to 700 mm). CCFL tubes provide a substantially even distribution of light and, therefore, have been used to provide background lighting for laptop computers and to provide light for scanning and copying. See, e.g., “A Cold Cathode Fluorescent Lamp (CCFL) Controller Used in Magnetic Transformer Application,” by Weiyun (Sophie) Chen, an article located on the internet at a web page having the address of http://www.chipcenter.com/analog/c070.htm (accessed Jun. 6, 2003).

CCFL tubes are small and portable, and provide high efficiency in light output. They also are effective in providing a substantially full spectrum of light, thereby facilitating effective therapy. Unit 50 may be relatively small. For example, the unit may be about six inches in length by five inches wide by two inches thick. The device typically provides intensities of 2,500 lux, 5,000 lux and 10,000 lux, which are adequate for most light therapy applications. Consequently, it is readily portable and may be used in travel, at the bedside and in many situations where larger units would be too intrusive.

Looking at FIG. 8, a perspective view is shown of the CCFL light unit 50 without a front cover. The cover for the embodiment of FIG. 8 is essentially the same as the cover shown in FIG. 3, including a lens for light output and a grill for the ion output. Six CCFL tubes 52 are placed in a generally parallel position relative to each other in a recess 54 of a housing 56. A generally parabolic reflector 58 is positioned behind each of tubes 52 for directing light toward the front of unit 50. Each tube 52 has electrical connections 60 and 62 extending from each end to connect to the power source (not shown). A high-density negative ion emitter device 64 includes a common plate 66 with individual ion plate emitters 68 attached thereto. Emitter device may be identical with the emitter device 44 shown in FIG. 5.

FIG. 9 shows the light unit 50 from a side view. Each of tubes 52 lies within the focal point of a parabolic portion 72 of the reflector 58. Reflector 58 rests on a circuit board 70 within housing 56, to which the tubes 52 and reflector 58 are operably attached. On the underside of circuit board 70 is an inverter 73 and a processor 74. A battery 76 is disposed just inside of circuit board 60. One of the emitter plates 68 is also shown. Each tube 52 is disposed at the focal point of a respective parabolic portion 72 of reflector 58. Accordingly, the rays of light (not shown) reflect in the same manner as shown in FIG. 7, to provide maximum reflectivity of the generated light.

The light therapy device of the embodiment shown in FIGS. 8 and 9 utilizes multiple cold cathode fluorescent technology for the treatment of light related problems, such as circadian rhythm problems and mood and sleep disorders. The light therapy device described herein provides long life (about 20,000 hours), substantially full-spectrum color while minimizing the presence of ultraviolet wavelengths.

Alternately, the light emitted by the CCFL tubes may be in the blue range (430-490 nm) and/or the green range (490-520 nm). The device also has a high CRI (Color Rendition Index), which is a measure of the trueness of color reflected when the light is exposed to a given color. In addition, the CCFL tubes of the present device include one lead on each end. They have a very small diameter, about the size of a plastic ink cylinder of a small writing pen.

The inverter 73 of the present embodiment may include a unit with the ability to dim down and ramp up the light output from the light source. One embodiment includes a dimming/ramping function built into the inverter. The dimming function enables a dusk simulation to aid in falling asleep, and the ramping function allows for natural waking.

Contrary to most uses of CCFL tubes, the high-intensity inverter of the present device is designed to run multiple CCFLs. This allows for fewer electronic components and thus lighter weight and smaller overall size of the unit.

The efficiencies of the CCFL technology allow the present device to be battery-powered. The device is designed to run on a multi-current wall transformer 120 volts or 240 volts, plus or minus 20%. The device may also contain rechargeable batteries with a capacity to allow multiple therapy sessions. The parabolic reflector unit 58 may be made of aluminum with a 95+% reflective coat. The reflector material is bent in a parabolic shape that insures that the light emitted from the tubes 52 is reflected forward to the user.

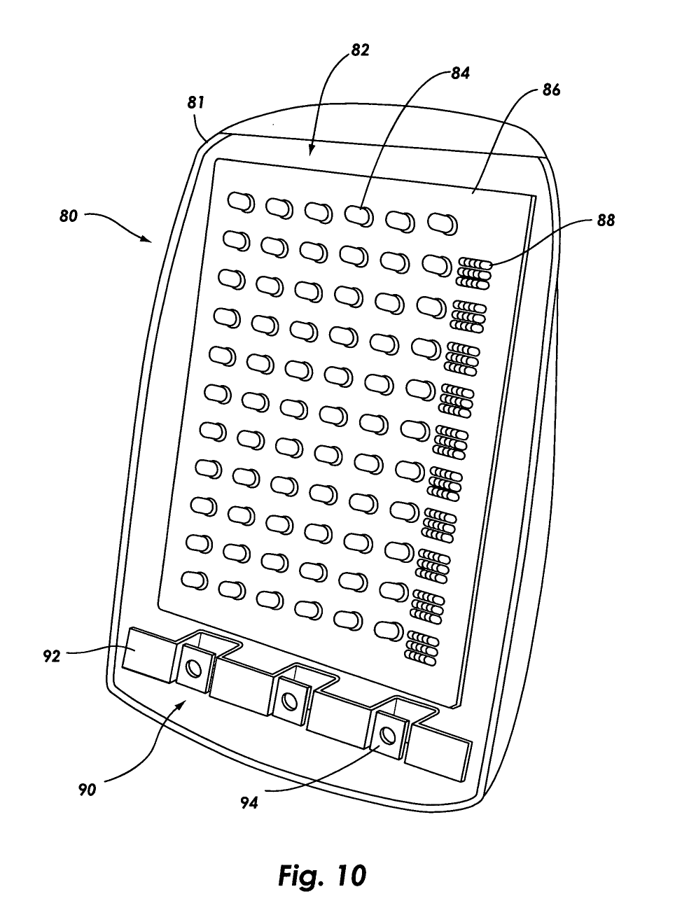

Referring now to FIG. 10, a therapy unit 80 is shown as a further embodiment of the present invention. In unit 80, the light source 82 comprises a matrix of light emitting diodes (LEDs) 84 mounted on a board 86 within a recess in housing 81. Electrical components 88 for operating the LEDs 84, such as resistors, are shown along the side of the LED light source 82. The front cover has been removed and is essentially the same as the front cover 15 shown in FIG. 3.

The LEDs 84 may be five millimeter oval LEDs emitting a selected spectrum of visible light. The light emission from light source matrix 52 may fall in an effective range of 10,000 lux to 12,000 lux at 6 to 12 inches. LEDs 84 may provide a full spectrum of light to a subject. Alternately, the LEDs 84 may provide a spectrum of light that is rich in what is called “blue light,” in the general range of 430-490 nm wavelength and particularly in the range of 454 to 464 nm wavelength. This range has sometimes been found to provide excellent suppression of melatonin to minimize the inducement of sleep. Alternately, the LEDs 84 may provide light in the green light range of 490-520 nm, alone or together with blue light. The LEDs 84 may provide useful treatment at distances of 15 to 30 inches with the most effective range being about 20 to 22 inches.

In all of the foregoing embodiments, the light and ion therapy timers are electronically operated with the data input buttons and display being configured essentially the same as that shown in FIG. 4. The timer setters for both the light therapy unit and the ion therapy unit may be set using the same three buttons 32 and the display window 30. The ionizer timing function may be set up to operate the ionizer portion independent of the light portion of unit 10. The light therapy may be conducted for a longer or shorter time than the ionizer therapy. This is possible because the combination of light and ionization therapy may work together to lower the total amount of time that the subject 12 (FIG. 1) may need, compared to therapy from light or ions alone. For example, in one embodiment, the subject 12 may want to operate the lamps 14 for about 20 to 30 minutes. In contrast, the ionizer unit may need to be activated for only about 15 to 20 minutes. Thus, data may be input on buttons 32 for operation of the light therapy portion for a longer period than the ion therapy portion.

Optionally, the subject or another individual may control the power supplied to, and thus, the intensity of radiation and concentration of ions respectively generated by the lamps or tubes and the ionizer. One control may be provided for use with both the lamps or tubes and the ionizer or the lamps or tubes may operate under influence of a separate control from that used to select the amount of power provided to the ionizer.

In summary, the present invention provides a number of advantages. The light therapy unit provides a beneficial spectrum of light, which may be a full spectrum of visible light or a light emphasizing or exclusively blue light. Alternately, the light output might emphasize or be exclusively green light, alone or together with blue light. Moreover, the therapy unit blocks out the ultraviolet rays. Using curved reflectors, the light output is substantially increased to provide a therapeutic intensity of at least about 10,000 lux at a distance of about six inches or more. The light therapy unit is integral with an ionization therapy unit and can be used in conjunction therewith. The ionization unit provides a high-density of negative ions to the subject, thereby enhancing mood and cleaning the air breathed by the subject.

The light therapy unit and ionization unit work together to provide beneficial therapy treatment and may together reduce the treatment time needed for a synergistic and beneficial therapy for a subject that is a substantial improvement over separate treatment by either light therapy or ion therapy alone. Not only is the time period for therapy substantially shortened, the combined therapies supplement each other to provide more effective therapy. In addition, having both therapies together makes it possible for each user to individually tailor the most effective combination of both light and ion therapy to fit his/her specific needs and physiological makeup. In addition, each unit is individually operable to achieve optimal light and ion therapy.

Although the above embodiments are representative of the present invention, other embodiments will be apparent to those skilled in the art from a consideration of this specification and the appended claims, or from a practice of the embodiments of the disclosed invention. It is intended that the specification and embodiments therein be considered as exemplary only, with the present invention being defined by the claims and their equivalents.

1. A light and ion therapy apparatus for delivering light to a subject to treat disorders that are responsive to ocular light therapy, comprising:
a light therapy unit for delivering to an eye of a subject light having an intensity of at least about 10,000 lux at a distance of at least about six inches or more; and
an ion therapy unit integral with the light therapy unit and independently operable in conjunction with or separate from the light therapy unit, to deliver to the subject a therapeutic dosage of high-density negative ions.

2. The light and ion therapy apparatus of claim 1, wherein the light therapy unit delivers to the subject light having an intensity of at least about 10,000 lux at a distance of at least about six to 15 inches for a period of 15 minutes or more.

3. The light and ion therapy apparatus of claim 1, wherein the ion therapy unit delivers to the subject high-density negative ions of about 2×10⁴ ions/cm³ or more.

4. The light and ion therapy apparatus of claim 1, wherein the light therapy unit includes a plurality of lamps, each having a curved reflector surrounding a portion of one of the plurality of lamps to direct light to the subject.

5. The light and ion therapy apparatus of claim 4, wherein each lamp of the plurality of lamps is substantially parabolic.

6. The light and ion therapy apparatus of claim 4, wherein each lamp of the plurality of lamps is disposed at a focal point of one of the curved reflectors.

7. The light and ion therapy apparatus of claim 1, wherein the light therapy unit is activated by a first timer and the ion therapy unit is activated by a second timer that operates independent of the first timer.

8. The light and ion therapy apparatus of claim 7, wherein the first timer may be set separately from the second timer to enable a user to tailor any desirable combination of light and ion therapy.

9. The light and ion therapy apparatus of claim 1, wherein the light therapy unit operates under control of a first selective power control and the ion therapy unit operates under control of a second selective power control.

10. The light and ion therapy apparatus of claim 9, wherein the first and second selective power controls are independently operable.

11. The light and ion therapy apparatus of claim 1, wherein the light therapy unit and the ion therapy unit together substantially reduce the treatment time needed for beneficial therapy relative to the amount of treatment time needed using the light therapy unit alone or the ion therapy unit alone.

12. The light and ion therapy apparatus of claim 1, wherein the light therapy unit and the ion therapy unit together provide a broader therapeutic effect than either the light therapy unit or the ion therapy unit could provide by itself.

13. The light and ion therapy apparatus of claim 1, wherein the light therapy unit delivers light to the subject substantially only in the blue light range.

14. The light and ion therapy apparatus of claim 1, wherein the light therapy unit delivers light to the subject having a greater intensity in the blue light range than of other wavelengths of light.

15. The light and ion therapy apparatus of claim 1, wherein the light therapy unit delivers light to the subject substantially only in the green light range.

16. The light and ion therapy apparatus of claim 1, wherein the light therapy unit delivers light to the subject having greater intensity in the green light range than of other wavelengths of light.

17. The light and ion therapy apparatus of claim 1, wherein the light therapy unit delivers no ultraviolet radiation.

18. The light and ion therapy apparatus of claim 1, further comprising a filter for restricting at least one wavelength of electromagnetic radiation delivered to the subject.

19. The light and ion therapy apparatus of claim 1, further comprising a diffraction element to increase a uniformity of a treatment field of light.

20. A light and ion therapy apparatus for delivering ocular light to a subject to treat disorders that are responsive to ocular light therapy, comprising:
a light therapy unit for delivering to an eye of a subject a therapeutic dosage of light including:
a plurality of lamps; and
a curved reflector surrounding a portion of at least one lamp of the plurality of lamps to direct light to the subject; and
an ion therapy unit integral with the light therapy unit for delivering to the subject a therapeutic dosage of high-density negative ions.

21. A light and ion therapy apparatus for delivering light to a subject to treat disorders that are responsive to ocular light therapy, comprising:
a light therapy unit for delivering a therapeutic dosage of light to an eye of a subject, and
an ion therapy unit integral with the light therapy unit for delivering to the subject a therapeutic dosage of high-density negative ions, wherein the ion therapy unit is activated independent of the light therapy unit, the ion dosage being delivered in conjunction with at least a portion of the light dosage.

22. A light and ion therapy method for delivering light to a subject to treat disorders that are responsive to ocular light therapy, comprising:
delivering to an eye of a subject light therapy comprising light having an intensity of at least about 10,000 lux at a distance of at least about six inches; and
independently delivering to the subject ion therapy comprising a therapeutic dosage of high-density negative ions, together with the light therapy or separate therefrom.

23. The light and ion therapy method of claim 22, wherein the light and ions are delivered to the subject at the same time, thereby substantially shortening the time needed for a therapeutic dose, compared to light therapy or ion therapy separately.

24. The light and ion therapy method of claim 22, wherein the light therapy unit and the ion therapy unit together provide a broader therapeutic effect than either the light therapy unit or the ion therapy unit could provide by itself.

25. The light and ion therapy method of claim 22, wherein the light is delivered by a plurality of lamps, further comprising individually reflecting the light from each lamp to increase the light delivery output.

26. The light and ion therapy method of claim 22, wherein the light is delivered in part with a curved light reflector adjacent to each lamp to reflect the light therefrom.

27. The light and ion therapy method of claim 26, further comprising disposing each of the plurality of lamps at a focal point of one of the curved light reflectors.

28. The light and ion therapy method of claim 26, wherein the reflector includes a parabolic curve.

29. The light and ion therapy method of claim 22, wherein the light is delivered to the eye of the subject at a distance of at least about six to 15 inches or greater.

30. The light and ion therapy method of claim 22, wherein light is delivered to the subject substantially only in the blue light range.

31. The light and ion therapy method of claim 22, wherein light is delivered to the subject having greatest intensity in the blue light range.

32. The light and ion therapy method of claim 22, wherein light is delivered to the subject substantially only in the green light range.

33. The light and ion therapy method of claim 22, wherein light is delivered to the subject having greatest intensity in the green light range.

34. The light and ion therapy method of claim 22, further comprising selectively filtering undesired wavelengths of electromagnetic radiation before light is delivered to the subject.

35. The light and ion therapy method of claim 22, wherein substantially no ultraviolet light is delivered to the subject.

36. The light and ion therapy method of claim 22, further comprising diffusing light to increase a uniformity of a treatment field including the delivered light.

37. The light and ion therapy method of claim 22, further comprising independently controlling times the light is delivered and the ions are delivered.

38. The light and ion therapy method of claim 22, further comprising independently controlling an intensity at which the light is delivered and a concentration of the ions that are delivered.

39. A light and ion therapy method for delivering ocular light to a subject to treat disorders that are responsive to ocular light therapy, comprising:
delivering to an eye of a subject a therapeutic dosage of light using a plurality of lamps, each having a curved reflector surrounding a portion of one of the plurality of lamps to direct light to the subject; and
delivering to the subject a therapeutic dosage of high-density negative ions.

40. A light and ion therapy method for delivering light to a subject to treat disorders that are responsive to ocular light therapy, comprising:
delivering a therapeutic dosage of light to the eyes a subject; and
independently delivering a therapeutic dosage of high-density negative ions to the subject in conjunction with delivering at least a portion of the dosage of light.
